(19) United States
(12) Patent Application Publication
(10) Pub. No.: US 2022/0279872 A1
(43) Pub. Date: Sep. 8, 2022

(54) WEARABLE AIRBAG DEVICE

(71) Applicant: TOYODA GOSEI CO., LTD., Kiyosu-shi (JP)

(72) Inventors: Toshihito YANAGISAWA, Kiyosu-shi (JP); Yusuke KAWAMURA, Kiyosu-shi (JP)

(21) Appl. No.: 17/674,166

(22) Filed: Feb. 17, 2022

(30) Foreign Application Priority Data
Mar. 5, 2021 (JP) 2021-35566

(51) Int. Cl. A41D 13/018; A41D 1/00; A41D 13/05; A61B 5/11; A61B 5/00

(52) U.S. Cl. CPC A41D 13/018; A41D 1/002; A41D 13/0506; A41D 13/0568; A61B 5/1117; A61B 5/6831; A61B 2562/0219

(57) ABSTRACT

There is provided a wearable airbag device that can protect a pelvic region of a wearer from a surface on which the wearer falls at the time of a fall. The device includes an airbag that is configured to be worn on the wearer around a pelvis, and to allow an inflation gas to flow thereinto and inflate to cover an outer side of a targeted body part formed of formed of left and right trochanters of femurs of the wearer. The airbag includes rolling suppression means for suppressing rolling on the surface on which the wearer falls at the time of coming into contact with the surface on which the wearer falls.

### CROSS REFERENCE TO RELATED APPLICATIONS

The present application claims priority from Japanese Patent Application No. 2021-35566 of Yanagisawa et al., filed on Mar. 5, 2021, the entire disclosure of which is incorporated herein by reference.

### BACKGROUND

### 1. Technical Field

The present invention relates to a wearable airbag device that can protect a pelvic region of a wearer from a surface on which the wearer falls at the time of a fall,

### 2. Description of Related Art

In the related art, as a wearable airbag device that can protect the pelvic region of a wearer (for example, an elderly person) at the time of a fall or the like, Pamphlet of International Publication No. 2019/207474 and JP-A-2008-22943 disclose a device configured to be worn on to be wrapped around the pelvic region, and cover the pelvic region with an airbag that inflates to protrude downward at the time of operation to protect the wearer from the surface on which the wearer falls such as the ground or floor.

In the wearable airbag device of the related art, the airbag that inflates to cover the pelvic region when the wearer falls is configured to be interposed between the surface on which the wearer falls such as the ground or floor and the pelvic region to protect the pelvic region. However, since the airbag allows the inflation gas to flow thereinto and inflates to make the surface curved, there is a case where the airbag rolls on the surface on which the wearer falls at the time of coming into contact with the surface on which the wearer falls. Therefore, there is room for improvement in that further rolling of the airbag is suppressed at the time of a fall and the wearer is accurately protected.

### SUMMARY

The present invention relates to a wearable airbag device having the following configuration.

There is provided a wearable airbag device that can protect a pelvic region of a wearer from a surface on which the wearer falls at the time of a fall. The device includes an airbag that is configured to be worn on the wearer around a pelvis, and to allow an inflation gas to flow thereinto and inflate to cover an outer side of a targeted body part formed of left and right trochanters of femurs of the wearer. The airbag includes rolling suppression means for suppressing rolling on the surface on which the wearer falls at the time of coming into contact with the surface on which the wearer falls.

### BRIEF DESCRIPTION OF DRAWINGS

FIG. 1 is a schematic view of a state where a wearable airbag device according to an embodiment of the present invention is worn on a wearer.

FIG. 2 is a plan view of a state where the wearable airbag device of the embodiment is laid flat.

FIG. 3 is a plan view of a state where an airbag to be used is laid flat in the wearable airbag device of the embodiment.

FIG. 4 is a sectional view of a state where the airbag of FIG. 3 has inflated by itself, and illustrates a part taken along taken along IV-IV of FIG. 3.

FIG. 5 is a schematic view of a state where the inflation of the airbag is completed in a state of being worn on the wearer in the wearable airbag device of the embodiment.

FIG. 6 is a schematic partial horizontal sectional view (a schematic horizontal sectional view on the left side) along a front-rear direction in a worn state illustrating a state where the inflation of the airbag is completed in the wearable airbag device of the embodiment.

FIG. 7 is a schematic vertical sectional view in a worn state illustrating a state where the inflation of the airbag is completed in the wearable airbag device of the embodiment.

FIG. 8 is schematic view describing a state where the wearer who is wearing the wearable airbag device of the embodiment falls,

FIG. 9 is a schematic sectional view illustrating a state where the wearer falls in a state where the airbag has inflated in the wearable airbag device of the embodiment.

FIG. 10 is a partial enlarged plan view illustrating an airbag of another embodiment of the present invention.

FIG. 11 is a sectional view of a state where the airbag of FIG. 10 has inflated by itself, and illustrates a part taken along XI-XI of FIG. 10.

FIG. 12 is a plan view of a tether used in the airbag of FIG. 10.

FIG. 13 is a schematic partial horizontal sectional view (a schematic horizontal sectional view on the left side) along the front-rear direction in a worn state illustrating a state where the inflation of the airbag of FIG. 10 is completed.

FIG. 14 is a schematic vertical sectional view in a worn state illustrating a state where the inflation of the airbag of FIG. 10 is completed.

FIG. 15 is a schematic sectional view illustrating an airbag according to still another embodiment of the present invention.

FIG. 16 is a schematic perspective view illustrating a state where the airbag, which is still another embodiment of the present invention, is inflated by itself.

FIG. 17 is a schematic horizontal sectional view of the airbag of FIG. 16,

FIG. 18 is a schematic vertical sectional view of the airbag of FIG. 16.

FIG. 19 is a plan view of a state where base members that configure the airbag of FIG. 16 are arranged side by side.

FIG. 20 is a schematic view in a worn state where the inflation of the airbag of FIG. 16 is completed.

FIG. 21 is a schematic partial horizontal sectional view (a schematic horizontal sectional view on the left side) along the front-rear direction in a worn state illustrating a state where the inflation of the airbag of FIG. 16 is completed.

FIG. 22 is a schematic vertical sectional view in a worn state illustrating a state where the inflation of the airbag of FIG. 16 is completed.

### DETAILED DESCRIPTION

Exemplary embodiments of the present disclosure are described below with reference to the accompanying drawings, However, the invention is not limited to the embodiments disclosed herein, All modifications within the appended claims and equivalents relative thereto are intended to be encompassed in the scope of the claims.

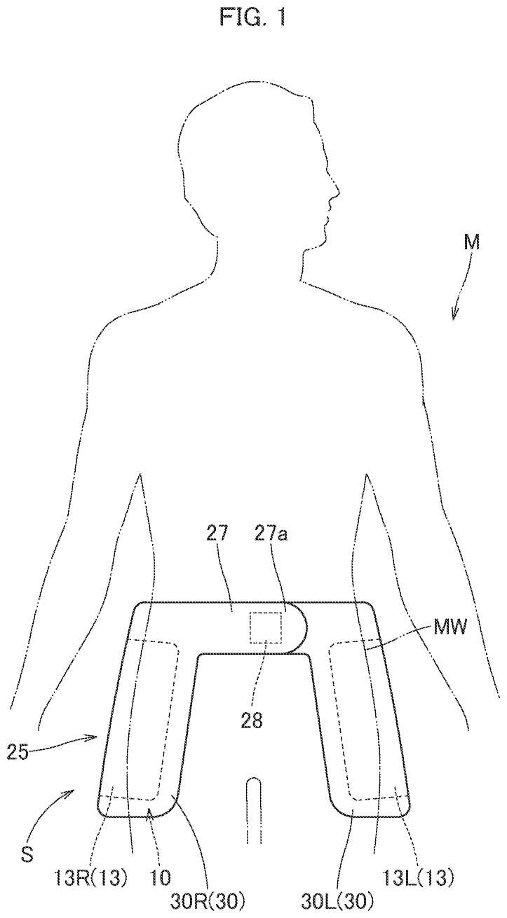

Hereinafter, one embodiment of the present invention will be described based on the drawings. As illustrated in FIG. 1, a wearable airbag device S according to the embodiment is configured to be worn by being wrapped around a pelvic region MW (specifically, around a pelvis MP) of a wearer M. Unless otherwise specified, up-down, front-rear, and left-right directions in the embodiment are intended to refer to up-down, front-rear, and left-right directions as viewed from the wearer M in a state of being worn on the wearer M.

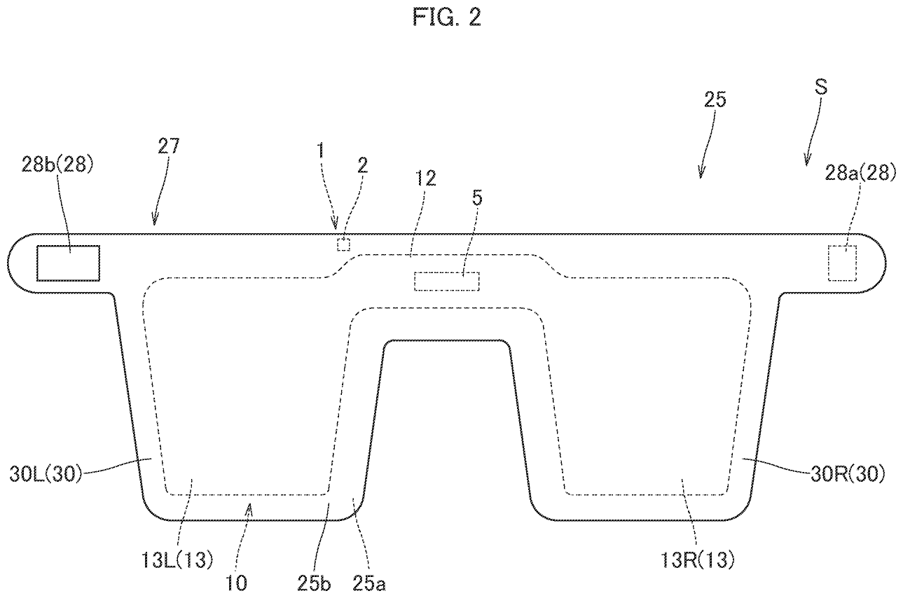

As illustrated in FIG. 2, the wearable airbag device S includes an airbag 10, a gas generator 5 for feeding the airbag 10 with an inflation gas, an operation control device 1 that includes a sensor part 2 for detecting a fall of the wearer M and operates the gas generator 5. and an outer cover 25 that covers an outer circumferential side of the airbag 10, In the wearable airbag device S according to the embodiment, as illustrated in FIGS. 1 and 2, the airbag 10 is disposed inside the outer cover 25 in a state of being laid flat.

The operation control device 1 includes the sensor part 2 including an angular velocity sensor capable of detecting angular velocities around three axes in the up-down, front-rear, and left-right directions, and an acceleration sensor capable of detecting accelerations in the three-axis directions. The operation control device 1 is configured to operate the gas generator 5 when a falling behavior different from a normal behavior of the wearer M is detected by the signal from the sensor part 2. Specifically, when the wearer M starts the falling behavior different from the normal behavior, the operation control device 1 includes determination means capable of making a determination from various threshold values, and thus the fall of the wearer M is detected based on the determination of the determination means, and the gas generator 5 is operated, The operation control device 1 further includes a power source formed of a battery (not illustrated) or the like for outputting signals for the operation of the sensor part 2 and for the operation of the gas generator 5.

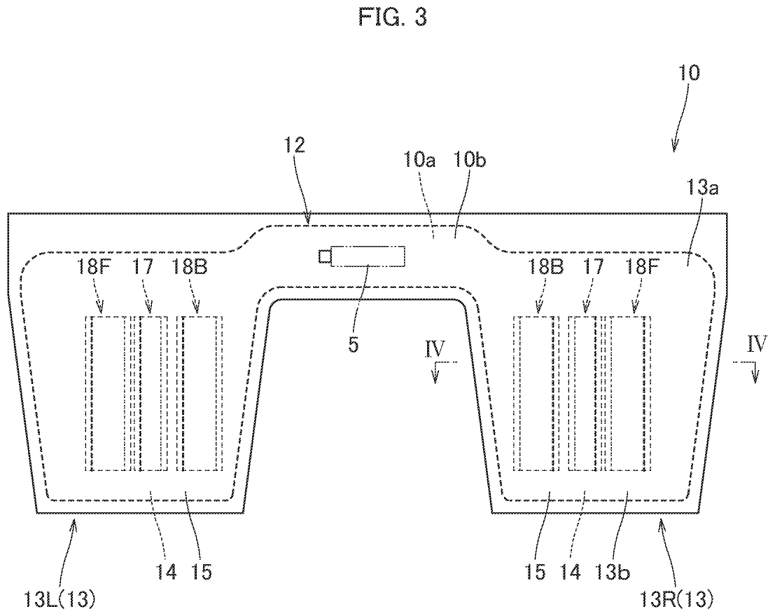

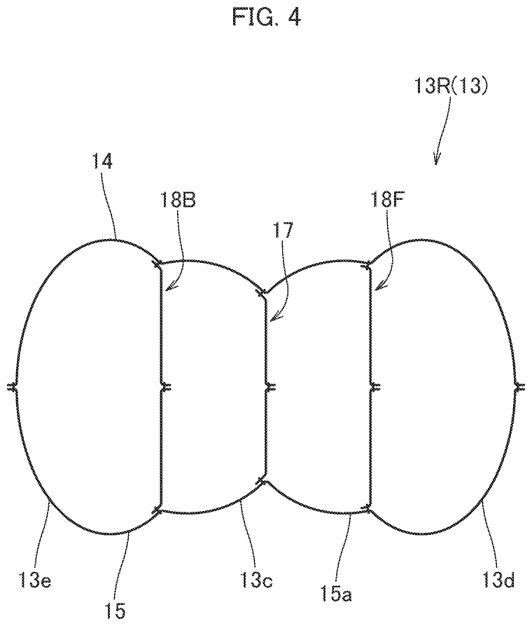

The airbag 10 is made of a sheet material having flexibility. In the case of the embodiment, the airbag 10 is made of a woven fabric made of polyester yarns, polyamide yarns or the like. The airbag 10 is configured to be worn on the wearer M around the pelvis MP by being wrapped around the wearer M at a wrap portion 27 (which will be described later) of the outer cover 25. In the case of the embodiment, the airbag 10 is disposed to cover left and right sides of the pelvic region MW of the wearer M in a worn state (refer to FIG. 1). As illustrated in FIGS. 3 and 4, the airbag 10 has an inner wall 10a disposed on the wearer M side and an outer wall 10b disposed on the outer side away from the wearer M at the time of wearing, with substantially the same external shape, The airbag 10 has a bag shape having a substantially plate-like inflation completed shape by sewing (joining) the circumferential edges of the inner wall 10a and the outer wall 10b to each other. In the case of the embodiment, as illustrated in FIG. 3, the airbag 10 includes two protecting inflation portions 13 (13L, 13R) that cover the left and right sides of the pelvic region MW of the wearer M when the inflation is completed, and a communication path 12 that is configured such that upper ends of the protecting inflation portions 13 (13L, 13R) communicate with each other, The airbag 10 is symmetrical in the left-right direction in a state of being laid flat.

The inflation completed shape of the communication path 12 is made in a substantially rod shape substantially along the left-right direction, and the communication path 12 is disposed in a region of the wrap portion 27 (which will be described later) in the outer cover 25. In the case of the embodiment, although the details are omitted in the drawing, the communication path 12 is disposed at a position which is a rear side of the pelvis MP of the wearer M when the inflation of the airbag 10 is completed. In the embodiment, the gas generator 5 is connected to the communication path 12 for feeding the airbag 10 with an inflation gas (refer to FIG. 2). Although the details are omitted in the drawing, the gas generator 5 is disposed in the vicinity of the center of the communication path 12 in the longitudinal direction. The gas generator 5 is configured to contain a compressed gas therein, and to release the contained state and discharge a cold gas into the airbag 10 when being operated. The gas generator 5 is configured to be electrically connected to the above-described operation control device 1, and to be operated with an operating signal input from the operation control device 1 that has detected the fall of the wearer M.

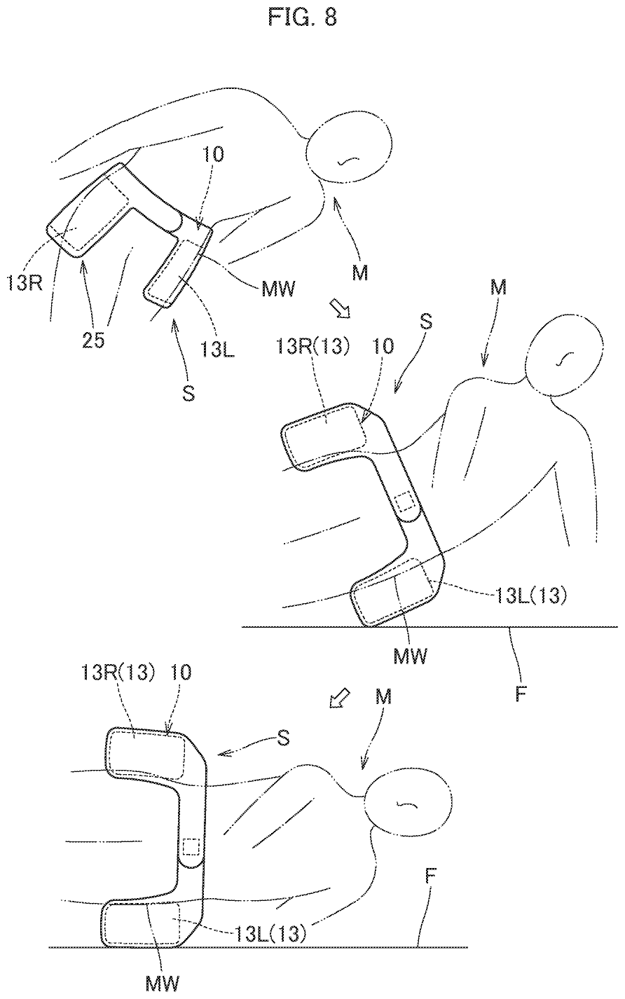

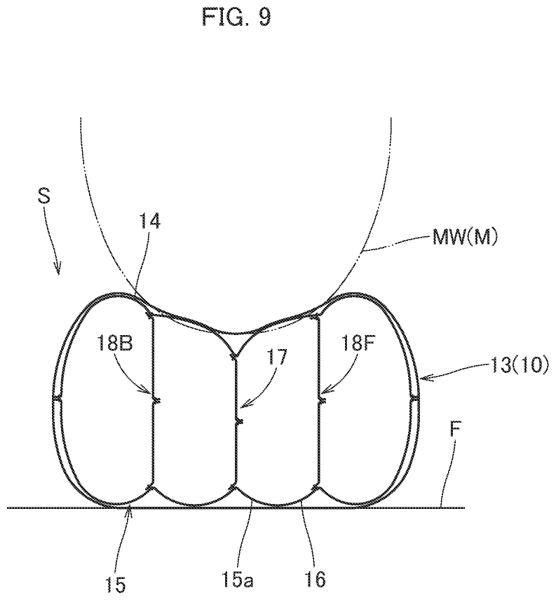

The protecting inflation portions 13 (13L, 13R) are disposed to have a step with respect to the communication path 12 such that an upper end 13a is positioned at a position slightly below the upper edge of the communication path 12 in a state where the airbag 10 is laid flat, and are formed so as to extend downward while extending to the left and right outsides from the communication path 12. Each of the protecting inflation portions 13 (13L, 13R) is configured to widely cover the surroundings (including the outer sides) of trochanters TP of femurs as targeted body parts at the time of wearing, and the external shape in a state of being laid flat is a substantially trapezoidal shape which is wide in the front-rear direction in the worn state and becomes slightly narrow as going toward the lower end side. Specifically, each of the protecting inflation portions 13 is configured to widely cover the region in the front-rear and up-down directions across the lower part (below the trochanter) of the trochanters TP of femurs from the sides of the pelvis MP to the wearer M when the inflation of the airbag 10 is completed. Further, each of the protecting inflation portions 13 is configured to be disposed substantially along the front-rear direction and substantially along the up-down direction, respectively, when the inflation is completed, on the outer surfaces 15a of the outer walls 15 disposed on the sides separated from the wearer M when the inflation is completed. Then, the outer surface 15a of the outer wall 15 configures rolling suppression means that comes into contact with a surface F on which the wearer M falls such as the ground or floor when the wearer falls as a planar contact surface 16 (refer to FIGS. 8 and 9), and can suppress the rolling of the protecting inflation portion 13,

Each of the protecting inflation portions 13 includes an inner wall 14 disposed on the wearer M side and an outer wall 15 disposed on the side separated from the wearer M when the inflation is completed, and is formed to loin the circumferential edges of the inner wall 14 and the outer wall 15 to each other. And, in the protecting inflation portion 13, the vertical tethers 17, 18F and 18B are arranged. Then, each of the protecting inflation portions 13 is configured such that the outer wall 15 can be disposed substantially along the front-rear direction and substantially along the up-down direction when the inflation is completed by providing the vertical tethers 17, 18F and 18B in the protecting inflation portion 13. In the case of the embodiment, the vertical tethers 17, 18F and 18B are arranged substantially along the up-down direction and are provided side by side at three places in the front-rear direction. As illustrated in FIG. 3, the three vertical tethers 17, 18F, and 18B are formed continuously except the upper end 13a side and the lower end 13b side of each of the protecting inflation portions 13 with substantially the same length dimension (in the up-down direction). Further, the vertical tethers 17, 18F, and 18B are configured to have different width dimensions. The central vertical tether 17 disposed on the center side in the front-rear direction has the smallest width dimension, and the width dimensions of the end vertical tethers 18F and 18B arranged on the end sides (both the front and rear sides of the central vertical tether 17) at the time of wearing are set to be larger than that of the central vertical tether 17 (refer to FIG. 4). The central vertical tether 17 is disposed substantially at the center (approximately at the center in the left-right direction in a state where the airbag 10 is laid flat) in the front-rear direction at the time of wearing, and the end vertical tethers 18F and 18B are arranged at positions that are substantially symmetrical with respect to the central vertical tether 17, and have substantially the same width dimension. In other words, in a state where each of the protecting inflation portions 13 inflates by itself in a state of being laid flat, each of the protecting inflation portions 13 is configured such that a central region 13c on the center side in the left-right direction (in the front-rear direction in the worn state) is made relatively thin along the up-down direction and the end regions 13d and 13e on both sides in the left-right direction (both sides in the front-rear direction in the worn state) are made relatively thick (refer to FIG. 4).

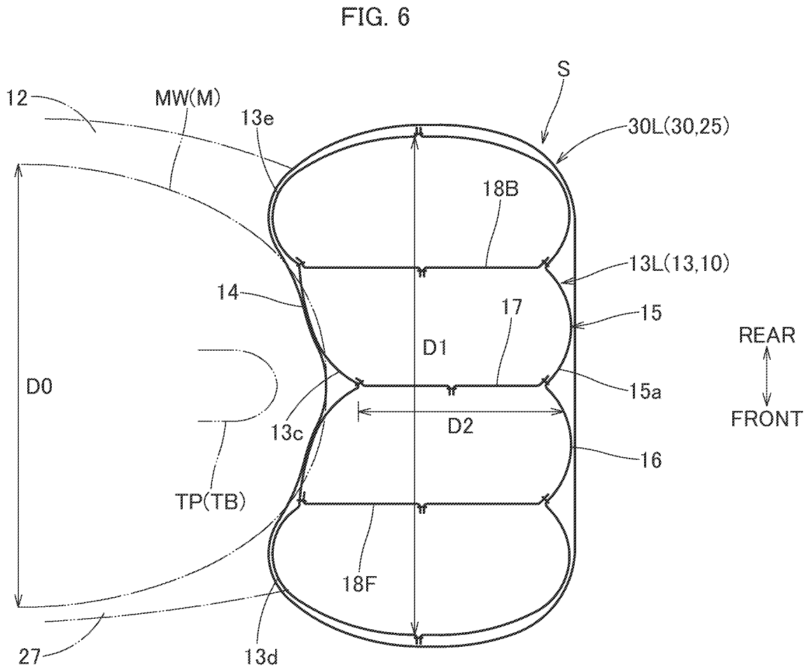

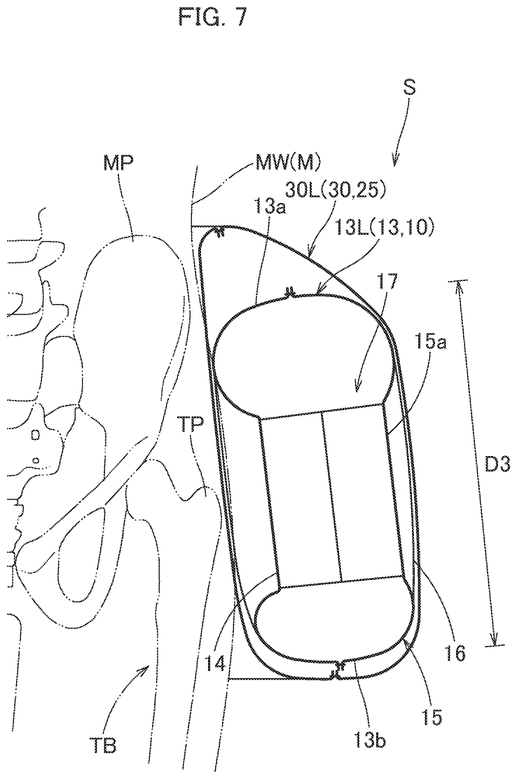

The airbag 10 is configured to be worn by being wrapped around the pelvis MP of the wearer M by using a wrap portion 27 (which will be described later) in the outer cover 25. In other words, when the inflation of the airbag 10 is completed, the communication path 12 is disposed to be curved along the curved state in the vicinity of the pelvis MP of the wearer M. The protecting inflation portion 13 disposed so as to extend downward from the communication path 12 is also configured to cover the region that curves in a substantially elliptical shape in the vicinity of the pelvic region MW of the wearer M, that is, on a cross section (cross section along the front-rear direction) along the horizontal plane (refer to FIG. 6). In a case where no tether is provided inside, the protecting inflation portion simply attempts to inflate in a shape having an elliptical cross section that is wide in the front-rear direction. However, in the airbag 10 of the embodiment, as described above, the protecting inflation portion 13 inflates such that the central region 130 at the center in the front-rear direction at the time of wearing is made relatively thin, and the end regions 13d and 13e on both sides in the front-rear direction are made thick. Therefore, when the thinnest central region 13c corresponds to the part positioned at the left and right outer ends on the side of the pelvic region MW, the protecting inflation portion 13 is disposed such that the inner wall 14 side is along the curve around the waist of the pelvic region MW and the outer wall 15 side is substantially along the front-rear direction in the cross section along the front-rear direction (refer to FIG. 6). Then, in each of the protecting inflation portions 13, the outer surface 15a of the outer wall 15 disposed substantially along the front-rear direction configures the planar contact surface 16, but the planar contact surface 16 is configured to connect the front edges to each other and the rear edges to each other, respectively, by the communication path 12 and the wrap portion 27 of the outer cover 25 (refer to the two-dot chain line in FIG. 6). Therefore, the planar contact surface 16 is stably formed into a planar shape when the inflation of the airbag 10 is completed. Specifically, the protecting inflation portion 13 is configured such that a width dimension D1 in the front-rear direction in a state where the inflation is completed is set to be at least larger than a width dimension D0 in the front-rear direction of the pelvic region MW of the wearer, and a thickness dimension D2 of the central region 13c is set to be approximately ½ of the width dimension D1 in the front-rear direction (refer to FIG. 6). Further, a width dimension D3 (refer to FIG. 7) in the up-down direction when the inflation of the protecting inflation portion 13 is completed is set to be slightly smaller than the width dimension D1 in the front-rear direction when the inflation is completed. The outer cover 25 is made of a woven fabric having an excellent flexibility that is more tactile than the base fabric configuring the airbag 10, and has an inner wall 25a disposed on the inside (on the wearer M side) at the time of wearing and an outer wall 25b disposed on the outer side at the time of wearing, with substantially the same external shape. The outer cover 25 has a bag shape by joining (sewing) the circumferential edges of the inner wall 25a and the outer wall 25b to each other. As illustrated in FIGS. 1, 2, and 5, the outer cover 25 has the substantially band-shaped wrap portion 27 disposed on the upper edge side and wrapped around the upper region of the pelvis MP of the wearer M, and two covering portions 30 (30L, 30R) which are formed to extend downward from the wrap portion 27 and cover the outer circumferential sides of each of the protecting inflation portions 13 (13L, 13R). A pair of hook-and-loop fasteners 28 as wearing means are provided on end portions 27a and 27b sides of the wrap portion 27. The hook-and-loop fastener 28 has a hook surface 28a and a loop surface 28b arranged on the end portions 27a and 27b sides of the wrap portion 27, respectively, and is configured to connect the end portions 27a and 27b of the wrap portion 27 to each other. The covering portions 30 (30L, 30R) are formed so as to extend downward from the wrap portion 27, and are respectively configured such that the external shape in a state of being laid flat is larger than the protecting inflation portion 13 and has a shape substantially similar to that of the protecting inflation portion 13 so as to make the protecting inflation portions 13 (13L, 13R) smoothly inflate inside.

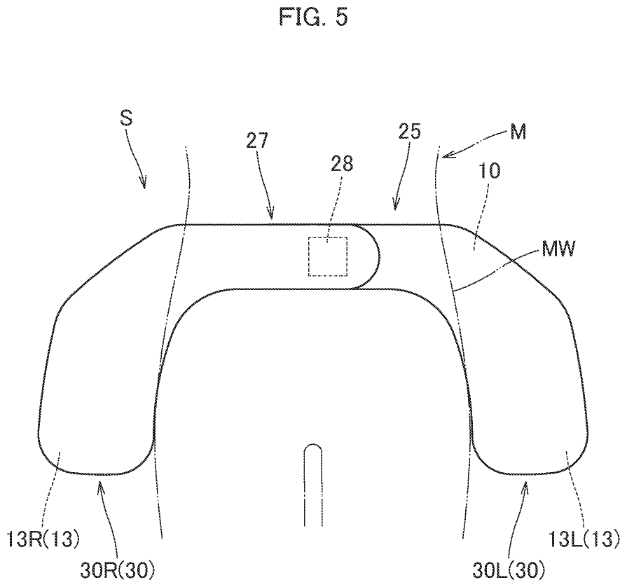

The wearable airbag device S of the embodiment is worn on the wearer M by being wrapped around the pelvic region MW (pelvis MP) of the wearer M by connecting the end portions 27a and 27b of the wrap portion 27 in the outer cover 25 to each other by using the hook-and-loop fastener 28 as wearing means (refer to FIG. 1), in addition, in the wearable airbag device S of the embodiment, in a state of being worn on the wearer M, when the sensor part 2 detects the fall of the wearer M as illustrated in FIG. 8, the operating signal is output to the gas generator 5 from the operation control device 1, the inflation gas flows into the airbag 10, and the airbag 10 completes the inflation as illustrated in FIGS. 5 to 7. Then, in the wearable airbag device S of the embodiment, in a state where the airbag 10 has inflated, when the wearer M fails such that the side of the pelvic region MW comes into contact with the surface F on which the wearer fails such as the ground or floor, one protecting inflation portion 13 (protecting inflation portion 13L on the left side in FIG. 8) on the surface F on which the wearer falls is interposed between the pelvic region MW and the surface F on which the wearer falls, and it is possible to protect the targeted body part including the trochanter TP of femur by the protecting inflation portion 13L.

Then, in the wearable airbag device S of the embodiment, the airbag 10 has the rolling suppression means (in the case of the embodiment, the planar contact surface 16 formed of the outer surface 15a of the outer wall 15 of the protecting inflation portion 13), and suppresses the rolling on the surface F on which the wearer falls when coming into contact with the surface F on which the wearer falls when the wearer M fails (refer to FIG. 9). Therefore, the airbag 10 can accurately protect the trochanter TP of femur as the targeted body part, and can also suppress damage in a case where the rolling of the airbag 10 causes the region of the wearer M that is not covered with the airbag to come into contact with the surface F on which the wearer falls, or the like.

Therefore, in the wearable airbag device of the embodiment, the wearer M can be accurately protected with a simple configuration.

Specifically, in the wearable airbag device S of the embodiment, the airbag 10 has two protecting inflation portions 13 (13L, 13R) that cover the outer sides of each of the trochanters TP of femurs when the inflation is completed, and is configured as the planar contact surface 16 such that the outer surfaces 15a of the outer walls 15 disposed on the side separated from the wearer M in each of the protecting inflation portions 13 are substantially along the front-rear direction and substantially along the up-down direction, respectively, when the inflation is completed. Then, the outer surface 15a (planar contact surface 16) configures the roiling suppression means, and when the wearer M falls, the outer surface 15a can come into contact with the surface F on which the wearer falls and suppress the rolling of the protecting inflation portion 13. In other words, in the wearable airbag device S of the embodiment, when the inflation of the airbag 10 is completed, the planar contact surfaces 16 formed of the outer surfaces 15a of the outer walls 15 in each of the protecting inflation portions 13 are disposed substantially along the front-rear and up-down directions. Therefore, when the wearer M falls, the planar contact surface 16 comes into contact with the surface F on which the wearer falls in a wide range, further rolling of the protecting inflation portion 13 can be accurately suppressed, and the protecting inflation portion 13 can stably protect the targeted body part (trochanter TP of femur) of the wearer M. The planar contact surface 16 (outer surfaces 15a of the outer walls 15 in each of the protecting inflation portions 13) is configured to connect the front edges to each other and the rear edges to each other, respectively, by the communication path 12 or the wrap portion 27 of the outer cover 25 (refer to the two-dot chain line in FIG. 6), and is formed to be stabilized when the inflation of the airbag 10 is completed.

More specifically, in the case of the embodiment, the protecting inflation portion 13 is configured to have the outer wall 15 and the inner wall 14 disposed on the wearer M side when the inflation is completed, and join the circumferential edges of the outer wall 15 and the inner wall 14 to each other. Further, the protecting inflation portion 13 is configured such that the outer surface 15a of the outer wall 15 when the inflation is completed is the planar contact surface 16 by providing the vertical tethers 17, 18F, and 18B connecting the outer wall 15 and the inner wall 14 inside. Therefore, the flat state of the outer wall can be appropriately set depending on the arranged part of the tethers, the number of arranged tethers, and the like, and the design can be easily changed. Specifically, in the airbag 10 of the embodiment, three vertical tethers 17, 18F, and 18B which are substantially along the up-down direction are provided side by side in the front-rear direction at the time of wearing, and by setting the width dimension of the central vertical tether 17 disposed on the center side to be smaller than the width dimensions of the end vertical tethers 18F and 18B arranged on both sides, the thickness when the inflation is completed is regulated. By changing the width dimensions of each of the vertical tethers 17, 18F, and 18B, the outer wall 15 is disposed substantially along the front-rear direction and substantially along the up-down direction at the time of wearing. In particular, in the airbag 10 of the embodiment, the protecting inflation portion 13 is set such that the width dimension D1 in the front-rear direction when the inflation is completed is set to be larger than the width dimension in the front-rear direction of the pelvic region MW of the wearer M, and the width dimension D3 in the up-down direction when the inflation is completed is set to be slightly smaller than the width dimension D1 in the front-rear direction, and accordingly, the side of the pelvic region MW is widely covered in the entire region in the front-rear and up-down directions. Therefore, at the time of a fall, the protecting inflation portion 13 comes into contact with the surface F on which the wearer falls on a surface wider in the front-rear direction than the pelvic region MW, and further rolling of the protecting inflation portion 13 can be stably regulated. Further, in the protecting inflation portion 13, the thickness dimension D2 of the central region 13c that is the thinnest and interposed between the trochanter TP of femur as a targeted body part and the surface F on which the wearer falls is set to approximately ½ of the width dimension D1 in the front-rear direction. Therefore, the trochanter TP of femur as a targeted body part can be protected stably with an excellent cushioning characteristic by the protecting inflation portion 13,

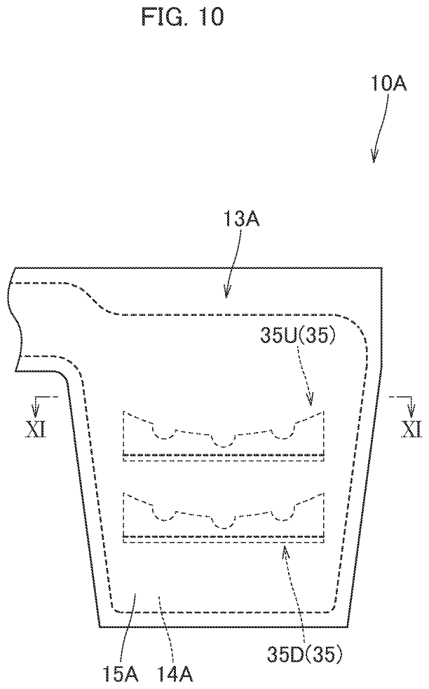

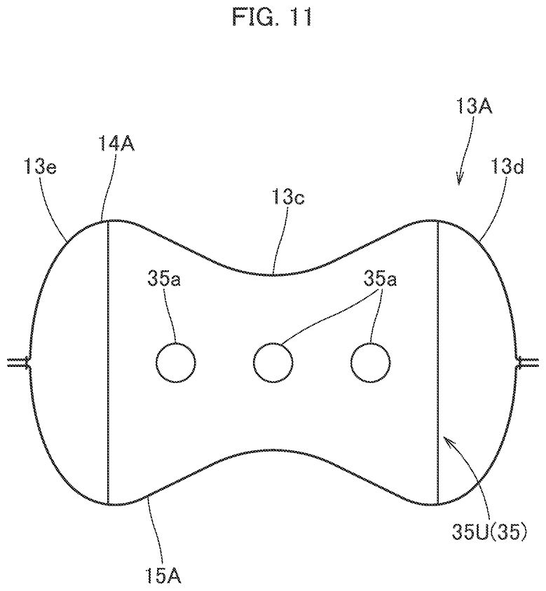

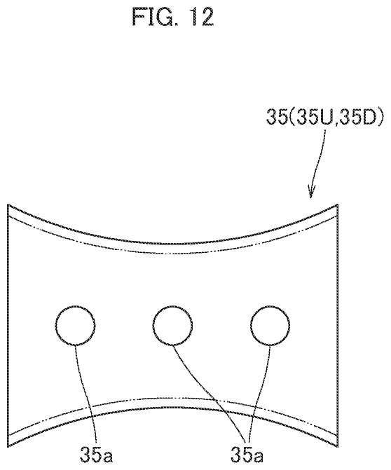

As an airbag 10A having a configuration in which the external shape of the protecting inflation portion when the inflation is completed is regulated by providing a tether inside, an airbag having the configuration illustrated in FIGS. 10 and 11 may be used. In the airbag 10A, in a protecting inflation portion 13A, as illustrated in FIGS. 10 and 11, when the inflation is completed in the worn state, the horizontal tethers 35 (35U, 35D) provided substantially along the horizontal plane (left-right direction in a state of being laid flat) are provided side by side at two places in the up-down direction. Each of the horizontal tethers 35 is provided except for the left edge side and the right edge side of the protecting inflation portion 13A in a state of being laid flat, and the circumferential edge is joined (sewn) to the outer wail 15A or the inner wall 14A over the entire length. Further, each of the horizontal tethers 35 is provided with a plurality (three in the case of the embodiment) of gas flow holes 35a through which the inflation gas can be inserted. In each of the horizontal tethers 35, the external shape in a state of being laid flat is a substantially concave lens shape such that the width dimension on the center side in the left-right direction is small and increases as going toward both ends (refer to FIG. 12).

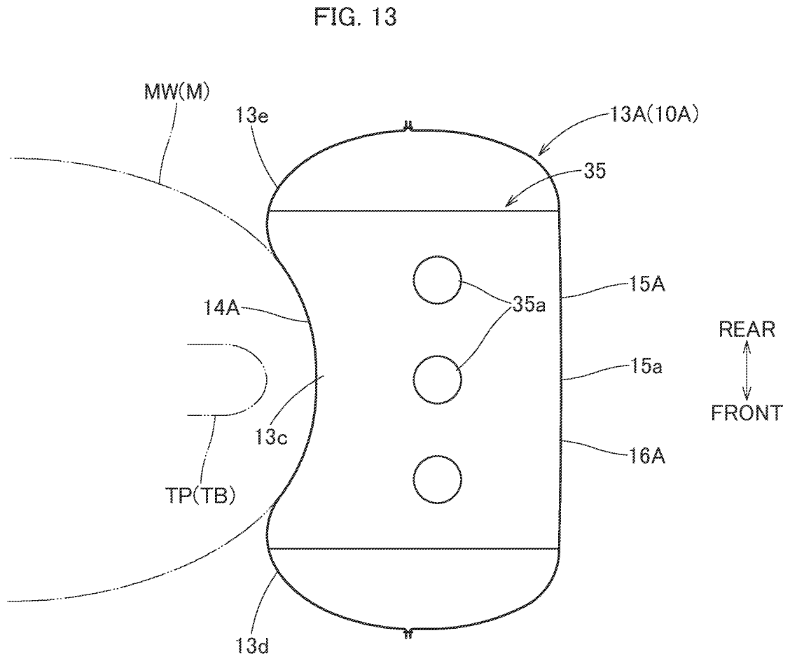

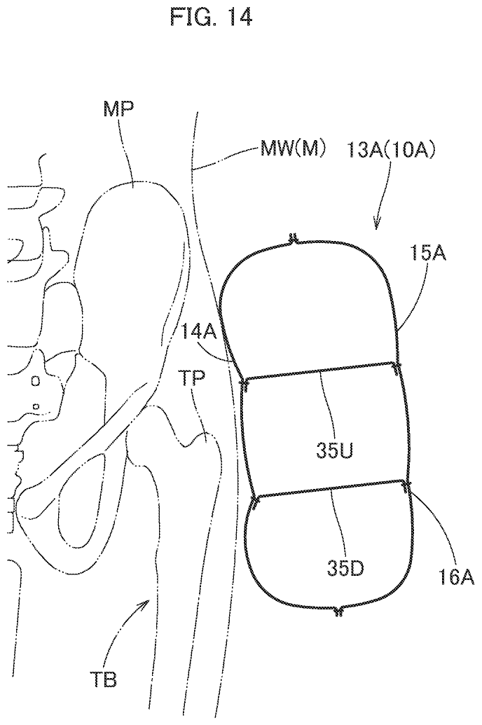

In a case of configuring the airbag 10A in this manner, in a state where the airbag 10A has inflated by itself, the protecting inflation portion 13A is configured such that the central region 13c on the center side in the left-right direction (in the front-rear direction in the worn state) is made relatively thin along the up-down direction and the end regions 13d and 13e on both sides in the left-right direction (both sides in the front-rear direction in the worn state) are made relatively thick. Therefore, in the worn state, the protecting inflation portion 13A is disposed such that the inner wall 14A side is along the curve around the waist of the pelvic region MW and the outer wall 15A side is substantially along the front-rear direction in the cross section along the front-rear direction (refer to FIG. 13). In other words, when the inflation of the airbag 10A is completed, the planar contact surfaces 16A formed of the outer surfaces 15a of the outer walls 15A of each of the protecting inflation portions 13A are disposed substantially along the front-rear and up-down directions. As a result, although the details are omitted in the drawing, when the wearer M falls, the planar contact surface 16A comes into contact with the surface on which the wearer falls in a wide range, further rolling of the protecting inflation portion 13A can be accurately suppressed, and the protecting inflation portion 13A can stably protect the targeted body part (trochanter TP of femur) of the wearer M. In FIGS. 13 and 14, the outer cover is omitted.

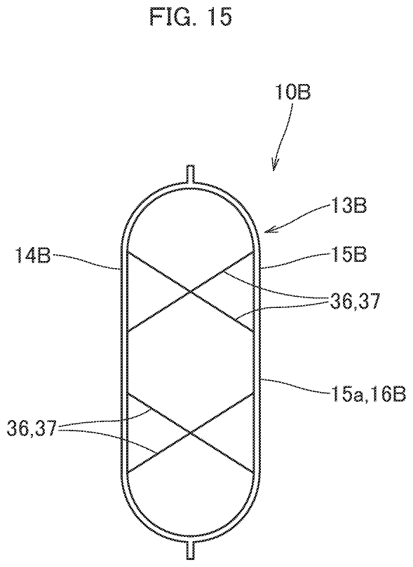

As the tethers provided inside the protecting inflation portions 13 and 13A, even when the plurality of vertical tethers 17, 18F, and 18B substantially along the up-down direction similar to the airbag 10 are provided side by side in the front-rear direction at the time of wearing, the plurality of horizontal tethers 35U and 35D substantially along the front-rear direction similar to the airbag 10A may be provided side by side in the up-down direction. In any configuration, the inflation completed shape is regulated such that the central region 13c is relatively thin and the end regions 13d and 13e on both sides in the front-rear direction are thick, and when the inflation is completed in the worn state, the outer surface 15a (planar contact surfaces 16 and 16A) of the outer walls 15 and 15A can be made flat. However, from the viewpoint of the workability (formation is possible by linear suturing work) at the time of joining the tether to the protecting inflation portion side and the thickness of the central region 13c that can be maintained to be wide and substantially constant in the up-down direction, similar to the airbag 10, it is preferable to provide the vertical tethers 17, 18F, and 18B which are substantially along the up-down direction inside the protecting inflation portion 13. The airbags 10 and 10A are formed into a bag shape by sewing (joining) the circumferential edges of a base fabric (inner wall 10a, outer wall 10b ) having a predetermined shape using suture threads. The airbag may be formed by bag weaving. In a case of being formed by bag weaving, as illustrated in FIG. 15, as an airbag 10B, for example, a configuration may be employed in which, inside a protecting inflation portion 13B, by crossing a warp thread 36 that configures one of an inner wall 14B or an outer wall 15B at the middle portion so as to configure the other one of the inner wall 14B or the outer wall 15B, and by providing a cross tether 37 while using the warp thread 36 as the cross tether 37, the shape of the outer wall 158 when the inflation is completed can be regulated. When the cross tethers 37 are provided at a plurality of places in the up-down direction or the front-rear direction, the outer wall 158 when the inflation is completed can be configured such that the outer surface 15a (planar contact surface 168) is made flat on a wide surface.

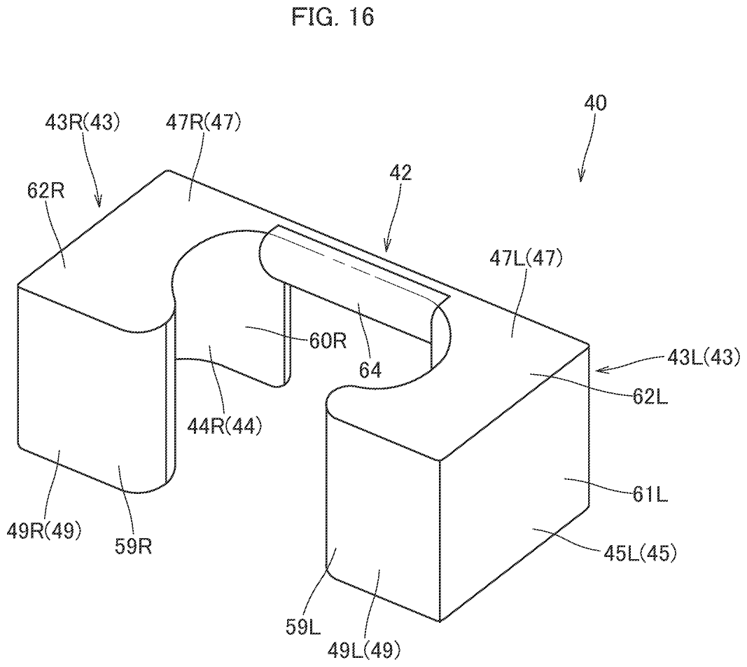

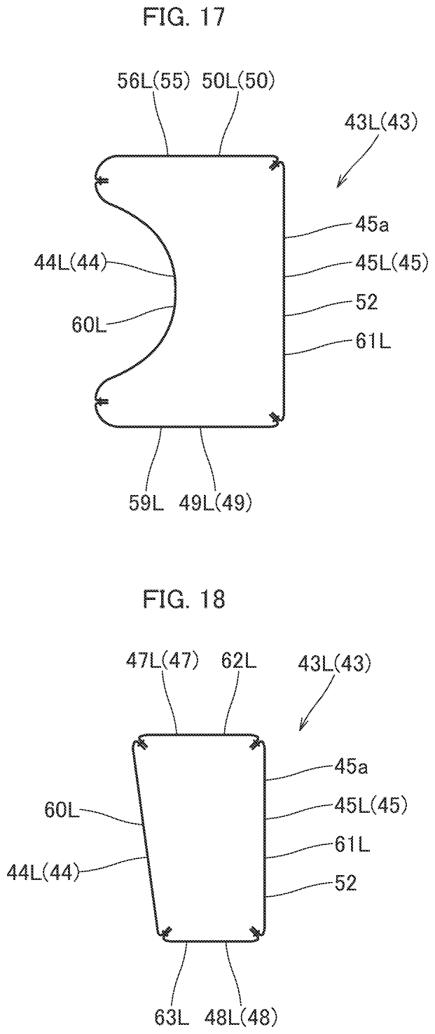

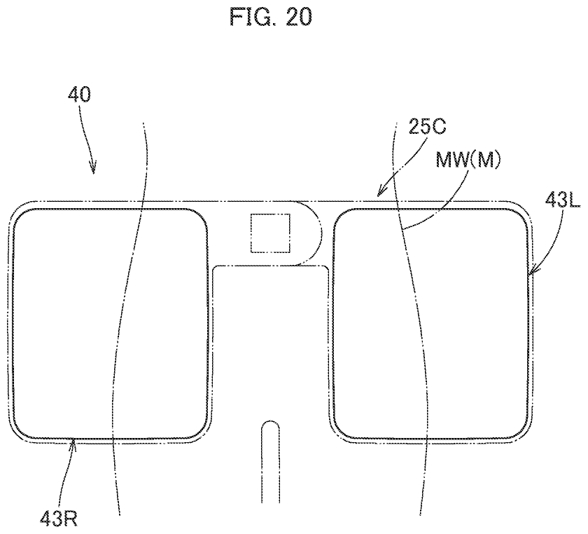

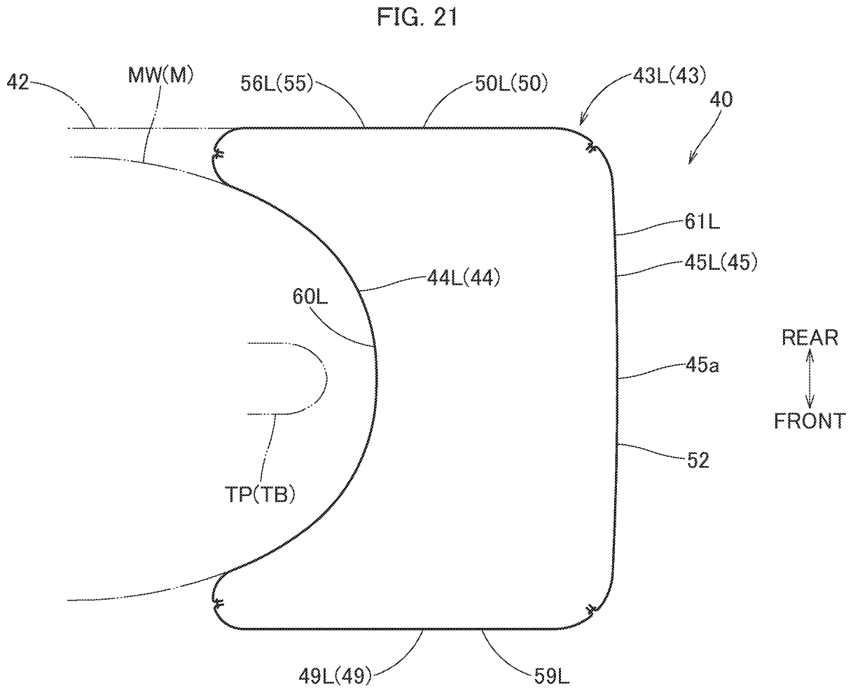

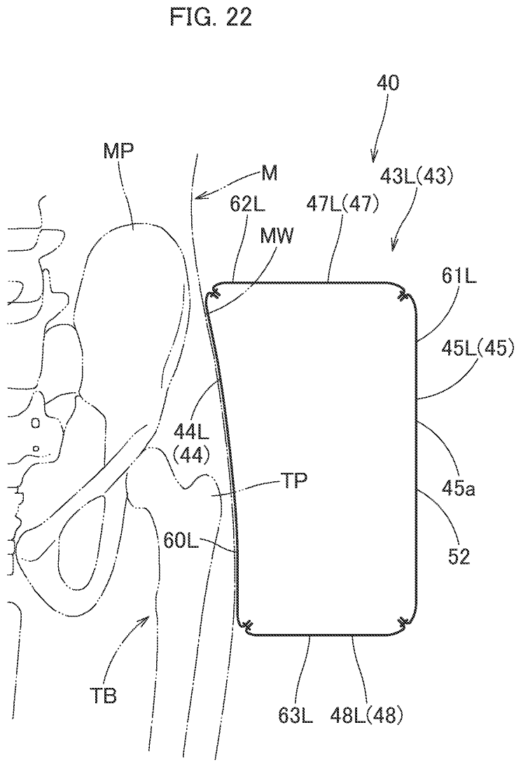

Furthermore, as the airbag 40, those having the configurations illustrated in FIGS. 16 to 22 may be used. Similar to the above-described airbag 10, the airbag 40 has two protecting inflation portions 43 (43L, 43R) that cover the left and right sides of the pelvic region MW of the wearer M when the inflation is completed, and a communication path 42 configured to communicate with the upper ends of the protecting inflation portion 43, As will be described later, the airbag 40 is formed by three-dimensionally joining the circumferential edges of a plurality of base members having a predetermined shape to each other. The airbag 40 is configured to be worn on the wearer M in a state of being accommodated in an outer cover 250 as illustrated by the two-dot chain line in FIG. 20, and although the details are omitted in the drawing, the airbag 40 inflates the inside of the outer cover 250. In the airbag 40 of this embodiment, each of the protecting inflation portions 43 (43L, 43R) has inner walls 44 (44L, 44R) disposed on the wearer M side when the inflation is completed, outer walls 45 (45L, 45R) disposed on a side separated from the wearer M, upper walls 47 (47L, 47R) and lower walls 48 (48L, 48R) disposed so as to connect between the inner wall 44 and the outer wall 45 and facing each other in the up-down direction when the inflation is completed, front walls 49 (49L, 49R) and rear walls 50 (50L, 50R) disposed so as to connect between the inner wall 44 and the outer wall 45 and facing each other in the front-rear direction in the worn state. In each of the protecting inflation portions 43 when the inflation is completed, the inner wall 44 is disposed so as to be curved according to the curved shape around the waist of the wearer M (refer to FIGS. 17 and 21). Further, the inner wall 44 is formed to be inclined in the up-down direction on the vertical section so as to expand downward (approaching the outer wall 45 across the lower end side) in accordance with the outward (left and right sides) protrusions from the pelvis MP of the wearer M to the vicinity of the trochanter TP of femur (refer to FIGS. 18 and 20). In each of the protecting inflation portions 43 when the inflation is completed at the time of wearing, the outer wall 45 is formed into a flat shape substantially along the front-rear and up-down directions. Then, in the airbag 40, the protecting inflation portion 43 disposes the outer wall 45 substantially along the front-rear direction and substantially along the up-down direction while the inner wall 44 is along the waist of the wearer M, and covers the left and right sides of the pelvic region MW, and the outer surface 45a of the outer wall 45 configures a planar contact surface 52 (refer to FIGS. 21 and 22).

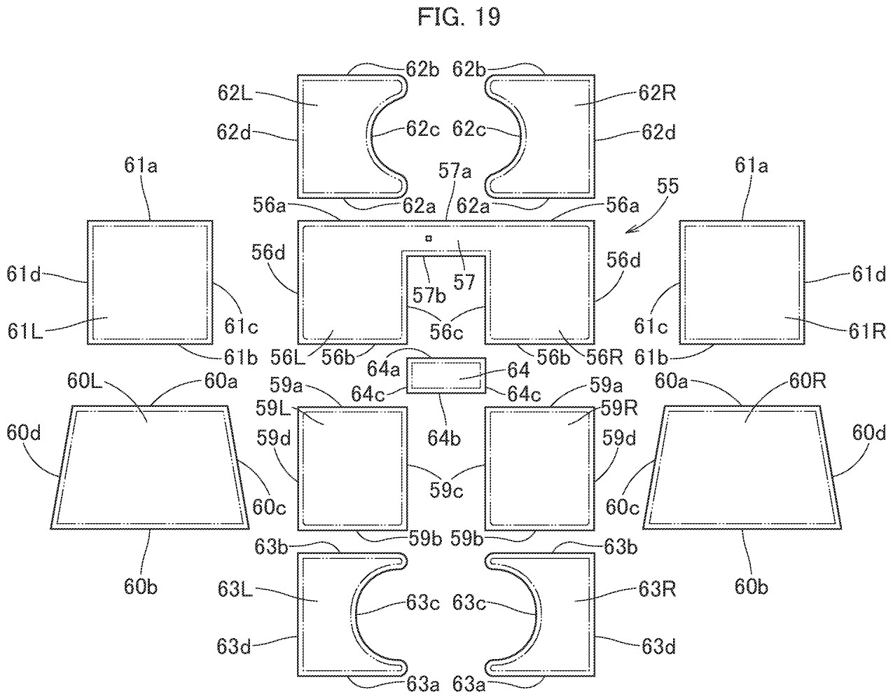

As illustrated in FIG. 19, the airbag 40 includes a total of 12 base members, such as a rear panel 55 that configures the rear walls 50L and 50R in each of the protecting inflation portions 43L and 43R and the outer wall 42b in the communication path 42, two front panels 59L and 59R that respectively configure the front walls 49L and 49R in each of the protecting inflation portions 43L and 43R, two inner panels 60L and 60R that configure the inner walls 44L and 44R in each of the protecting inflation portions 43L and 43R, two outer panels 61L and 61 R that configure the outer walls 45L and 45R in each of the protecting inflation portions 43L and 43R, two upper panels 62L and 62R that configure the upper walls 47L and 47R in each of the protecting inflation portions 43L and 43R, two lower panels 63L and 63R that configure the lower walls 48L and 48R in each of the protecting inflation portions 43L and 43R, and a communication path panel 64 that configures the inner wall 42a in the communication path 42. The airbag 40 is formed in a bag shape by three-dimensionally joining (sewing) the circumferential edges corresponding to the rear panel 55, the front panels 59L and 59R, the inner panels 60L and 60R, the outer panels 61L and 61 R, the upper panels 62L and 62R, the lower panels 63L and 63R, and the communication path panel 64. The rear panel 55 includes rear wall configuration portions 56L and 56R that respectively configure the rear walls 50L and 50R of each of the protecting inflation portions 43, and a communication passage configuration portion 57 that configures the outer wall 42b of the communication path 42. The inner panels 60L and 60R that configure the inner wall 44 of each of the protecting inflation portions 43 have a substantially trapezoidal shape that is wide across a lower edge 60b side. Further, in each of the protecting inflation portions 43, the upper panels 62L and 62R that configure the upper wall 47 and the lower panels 63L and 63R that configure the lower wall 48. The inner edges 62c and 63c are formed to be curved to form a curved shape of the inner wall 44.

Specifically, the airbag 40 of the embodiment is configured as follows. In the rear panel 55, an upper edge 56a of each of the rear wall configuration portions 56L and 56R is joined to a rear edge 62b of each of the upper panels 62L and 62R, a lower edge 56b is joined to a rear edge 63b of each of the lower panels 63L and 63R, an inner edge 56c is joined to a rear edge 60d of the inner panels 60L and 60R, and an outer edge 56d is joined to a rear edge 61d of the outer panels 61L and 61 R. The upper edge 57a and the lower edge 57b of the communication passage configuration portion 57 are joined to the upper edge 64a and the lower edge 64b of the communication path panel 64, respectively. An upper edge 59a of each of the front panels 59L and 59R is joined to a front edge 62a of each of the upper panels 62L and 62R, a lower edge 59b is joined to a front edge 63a of each of the lower panels 63L and 63R, an inner edge 59c is joined to a front edge 60c of each of the inner panels 60L and 60R, and an outer edge 59d is joined to a front edge 61c of each of the outer panels 61L and 61R. The upper edge 60a of each of the inner panels 60L and 60R is joined to the inner edge 62c of each of the upper panels 62L and 62R, and the lower edge 60b is joined to the inner edge 63c of each of the lower panels 63L and 63R. The upper edge 61a of each of the outer panels 61L and 61R is joined to the outer edge 62d of each of the upper panels 62L and 62R, and the lower edge 61b is joined to the outer edge 63d of each of the lower panels 63L and 63R. A side edge 64c of the communication path panel 64 is joined to the vicinity of the rear end of the inner edge 62c in each of the upper panels 62L and 62R and the vicinity of the upper end of the rear edge 60d in each of the inner panels 60L and 60R.

Even in the airbag 40 having such a configuration, when the inflation is completed, the planar contact surface 52 formed of the outer surfaces 45a of the outer walls 45 of each of the protecting inflation portions 43 is disposed substantially along the front-rear and up-down directions as illustrated in FIGS. 20 and 21, and accordingly, when the wearer M falls, the planar contact surface 52 comes into contact with the surface on which the wearer falls (not illustrated) in a wide range, and further rolling of the protecting inflation portion 43 can be accurately suppressed. Therefore, the protecting inflation portion 43 can stably protect the targeted body part (trochanter IP of femur) of the wearer M. Further, although the airbag 40 having the above-described configuration is configured to three-dimensionally join the circumferential edges of the base member, the number of tethers to be disposed inside can be suppressed (in the case of the embodiment, the tethers are not disposed inside), and a simple configuration is possible. In FIGS. 20 and 21, the outer cover is omitted. in the wearable airbag device S of the embodiment, the vicinity of the base of the femur TB of the wearer M (trochanter TP of femur) can be protected by the two protecting inflation portions 13, 13A, and 43 of the airbags 10, 10A, and 40. Therefore, the wearable airbag device S can suppress the fracture of the femur TB of the wearer M, which is caused by the fall and for which the treatment is prolonged, and be suitably used for the elderly person.

In the embodiment, as the wearable airbag device S, a configuration is described as an example, in which the airbags 10, 10A, and 40, which are worn to be wrapped around the pelvic region MW (pelvis MP) and accommodated in a state of being laid flat inside the outer cover 25, inflate to protect the pelvic region MW (trochanter TP of femur) of the wearer M. The wearable airbag device to which the present invention is applicable is not limited to the embodiment. The present invention is also applicable to a type of wearable airbag device in which the airbag is accommodated inside the outer cover in a folded state and is worn to be wrapped around the pelvic region (pelvis). The present invention is also applicable to a type of wearable airbag device, such as a vest or a jacket, which is worn on the torso of the wearer and inflates by protruding the airbag so as to protect the pelvic region from the lower end side in the worn state.

The present invention relates to a wearable airbag device having the following configuration.

There is provided a wearable airbag device configured to protect a pelvic region of a wearer from a surface on which the wearer falls, the device including: an airbag configured to be worn on the wearer around a pelvis, and to allow an inflation gas to flow thereinto and inflate to cover an outer side of a targeted body part formed of left and right trochanters of femurs of the wearer, in which the airbag includes rolling suppression means for suppressing rolling on the surface on which the wearer falls at the time of coming into contact with the surface on which the wearer falls.

In the wearable airbag device of the present invention, the airbag includes the rolling suppression means and the rolling on the surface on which the wearer falls is suppressed when the airbag comes into contact with the surface on which the wearer falls when the wearer falls. Therefore, the airbag can accurately protect the targeted body part, and can also suppress damage in a case where the rolling of the airbag causes the region of the wearer that is not covered with the airbag to come into contact with the surface on which the wearer falls, or the like.

Therefore, the wearable airbag device of the present invention can accurately protect the wearer at the time of a fall.

Specifically, in the wearable airbag device of the present invention, it is preferable that the airbag have two protecting inflation portions that cover the outer sides of each of the targeted body parts when inflation is completed, and use outer surfaces of outer walls disposed on sides separated from the wearer in each of the protecting inflation portions as planar contact surfaces respectively configured to be disposed substantially along a front-rear direction and substantially along an up-down direction when the inflation is completed, and the planar contact surface configure the rolling suppression means and be configured to come into contact with the surface on which the wearer falls when the wearer falls and to suppress rolling of the protecting inflation portion.

According to the above-described configuration of the wearable airbag device, when the inflation of the airbag is completed, the planar contact surface formed of the outer surfaces of the outer walls of each of the protecting inflation portions is disposed substantially along the front-rear and up-down directions, and accordingly, when the wearer falls, the planar contact surface will come into contact with the surface on which the wearer falls in a wide range. Therefore, further rolling of the protecting inflation portion can be accurately suppressed, and the targeted body part of the wearer can be stably protected by the protecting inflation portion.

Furthermore, specifically, a configuration can be employed in which the protecting inflation portion has the outer wall and an inner wall disposed on the wearer side when the inflation is completed, joins circumferential edges of the outer wall and the inner wall to each other, and is provided with a tether that connects the outer wall and the inner wall on an inside thereof such that the outer surface of the outer wall when the inflation is completed is used as the planar contact surface. With such a configuration, the flat state of the outer wall can be appropriately set depending on the arranged part of the tethers, the number of arranged tethers, and the like, and the design can be easily changed.

Further, the outer circumferential wall of the airbag may be formed of the plurality of base members, and the protecting inflation portion having the planar contact surface may be formed by three-dimensionally joining the circumferential edges of the base members. With such a configuration, the number of tethers to be disposed inside can be suppressed, and the airbag can be easily configured.

What is claimed is:

1. A wearable airbag device configured to protect a pelvic region of a wearer from a surface on which the wearer falls, the device comprising:
an airbag configured to be worn on the wearer around a pelvis, and to allow an inflation gas to flow thereinto and inflate to cover an outer side of a targeted body part formed of left and right trochanters of femurs of the wearer, wherein
the airbag includes rolling suppression means for suppressing rolling on the surface on which the wearer falls at the time of coming into contact with the surface on which the wearer falls.

2. The wearable airbag device according to claim 1, wherein
the airbag has two protecting inflation portions that cover the outer sides of each of the targeted body parts when inflation is completed, and uses outer surfaces of outer walls disposed on sides separated from the wearer in each of the protecting inflation portions as planar contact surfaces respectively configured to be disposed substantially along a front-rear direction and substantially along an up-down direction when the inflation is completed, and
the planar contact surface configures the rolling suppression means and is configured to come into contact with the surface on which the wearer falls when the wearer falls and to suppress rolling of the protecting inflation portion.

3. The wearable airbag device according to claim 2, wherein
the protecting inflation portion has the outer wall and an inner wall to be disposed on a wearer side when the inflation is completed, is formed by joining circumferential edges of the outer wall and the inner wall to each other, and is provided with a tether that connects the outer wall and the inner wall on an inside thereof such that the outer surface of the outer wall when the inflation is completed is used as the planar contact surface.

4. The wearable airbag device according to claim 3, wherein
the tether is formed of three vertical tethers which are arranged substantially along the up-down direction and provided side by side in the front-rear direction when the inflation of the airbag is completed in a worn state, and
a width dimension of a central vertical tether disposed on a center side is set to be smaller than those of end vertical tethers arranged on both front and rear sides. cm 5. The wearable airbag device according to claim 3, wherein
the tether is formed of a plurality of horizontal tethers which are arranged substantially along the front-rear direction and provided side by side in the up-down direction when the inflation of the airbag is completed in a worn state, and
the horizontal tether has an external shape in a state of being laid flat which is a concave lens shape such that a width dimension on a center side is small and increases as going toward both end sides.

6. The wearable airbag device according to claim 3, wherein
the airbag is formed by bag weaving and,
the tethers are formed by crossing warp threads that form one of the inner wall or the outer wall at a middle portion so as to form the other one of the inner wall or the outer wall, to be formed at a plurality of places.

7. The wearable airbag device according to claim 2, wherein
an outer circumferential wall of the airbag is formed of formed of a plurality of base members, and
the protecting inflation portion having the planar contact surface three-dimensionally joins circumferential edges of the base members.

8. The wearable airbag device according to claim 2, wherein
the device is configured to be worn on the wearer by being wrapped around the pelvic region, and
the airbag covers an outer circumferential side with an outer cover.
